(19) United States
(12) Patent Application Publication
(10) Pub. No.: US 2026/0340842 A1
(43) Pub. Date: Oct. 29, 2026

(54) IMAGE RECONSTRUCTION METHOD FOR A MEDICAL SYSTEM

(71) Applicant: medPhoton GmbH, Salzburg (AT)

(72) Inventors: Heinz DEUTSCHMANN, Salzburg (DE); Philipp STEININGER, Salzburg (DE); Felix GINZINGER, Salzburg (DE); Joachim SPIELBICHLER, Salzburg (DE); Julia COSTE-MARIN, Salzburg (DE); Anastasiia PARII, Salzburg (DE); Malte LANGKABEL, Salzburg (DE); Daniel KELLNER, Salzburg (DE)

(21) Appl. No.: 19/099,122

(22) PCT Filed: Mar. 20, 2024
(86) PCT No.: PCT/EP2024/057476

§ 371 Date: Jan. 28, 2025

(30) Foreign Application Priority Data
Mar. 21, 2023 (WO) PCT/EP2023/057220

(51) Int. Cl. G06T 7/246; G06T 7/55; G16H 30/40

(52) U.S. Cl. CPC G06T 7/248; G06T 7/55; G16H 30/40; G06T 2207/10016; G06T 2207/30164

(57) ABSTRACT

Provided is an image reconstruction method and a medical system for image reconstruction. The medical system includes a medical imaging system, and a tracking device attached to the medical imaging system. The method includes calibrating a coordinate system C2 of the tracking device for marker-based tracking to a coordinate system C1 of the medical imaging system; and performing, via the medical imaging system, a scan, wherein performing the scan includes acquiring a plurality of medical images I_i (i=1 . . . n). The method further includes acquiring, for each of the plurality of medical images I_i and via the tracking device, corresponding tracking coordinates in C2 using one or more markers; and transforming the medical images based on the tracking coordinates and the calibration between the coordinate systems C1 and C2. Additionally, the method includes reconstructing, based on the transformed images, 3D images with respect to a reference position.

### FIELD OF THE INVENTION

The present invention relates to an image reconstruction method for a medical system, a corresponding computer program product and computer-readable medium, and a (medical) system.

### TECHNICAL BACKGROUND

In the context of medical imaging, motion artifacts present a general problem. Therefore, tracking is often employed to detect motion. Detected motion can be used to reduce motion artifacts. Known approaches require a high degree of periodicity in the motion pattern. A subject-motion might be tracked using a stationary tracking device.

However, medical imaging systems having more degrees of freedom are now being used. One example is the Loop-X system being freely movable on wheels and having a tiltable gantry. The additional degrees of freedom are very challenging in terms of image reconstruction, particularly motion compensation, as the position of the imaging system at the time of capturing each respective image and motion of a subject during a scan needs to be taken into account.

Known systems and methods are prone to various motion artifacts in such challenging scenarios.

The present invention, accordingly, has the object of providing improved image quality with reduced motion artifacts.

The present invention can be used for medical imaging, particularly allowing for periodic or non-periodic motion of the imaged subject and/or the imaging device, e.g. in connection with a Loop-X imaging system or navigation in medical procedures and/or together with systems for image-guided radiotherapy such as VERO® and ExacTrac®, both products of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The invention provides an image reconstruction method for a medical system comprising a medical imaging system and a tracking device, e.g. attached to the medical imaging system, the method comprising calibrating a coordinate system C2 of the tracking device for marker-based tracking to a coordinate system C1 of the medical imaging system; performing a scan by means of the medical imaging system, wherein performing the scan comprises acquiring a plurality of medical images I_i (i=1 . . . n); by means of the tracking device, for each of the plurality of medical images I_i, acquire corresponding tracking coordinates in C2 using one or more markers; transforming the medical images based on the tracking coordinates and the calibration between C1 and C2 ; and reconstructing 3D images based on the transformed images with respect to a reference position.

### GENERAL DESCRIPTION OF THE INVENTION

In this section, a description of the general features of the present invention is given for example by referring to possible embodiments of the invention.

The invention provides an image reconstruction method, medical system, computer program product, and computer readable medium according to the independent claims. Preferred embodiments are laid down in the dependent claims. The invention provides an image reconstruction method for a medical system comprising a medical imaging system and a tracking device rigidly attached, to the medical imaging system, the method comprising calibrating a coordinate system C2 of the tracking device for marker-based tracking to a coordinate system C1 of the medical imaging system; performing (S12) a scan by means of the medical imaging system, wherein performing the scan comprises acquiring a plurality of medical images I_i (i=1 . . . n); by means of the tracking device, for each of the plurality of medical images I_i, acquire corresponding tracking coordinates in C2 using one or more markers; transforming the medical images based on the tracking coordinates and the calibration between C1 and C2 ; and reconstructing 3D images based on the transformed images with respect to a reference position, which may for example be associated with a reference medical image.

Thus, motion as detected in C2 based on the tracking coordinates is accounted for during the 3D image reconstruction making use of the calibration between C1 and C2 and a reference position (and corresponding medical image) with respect to which motion is compensated.

The claimed method is highly reliable and efficient in reducing motion artefacts.

The method is suitable for periodic and non-periodic movement.

For example, other approaches might use detected motion to reduce motion artifacts by gating image scanning (i.e. interrupt scanning when motion occurs und resume scanning when motion stops and is back at position where scanning was interrupted) or by binning the acquired raw images into different phases of motion (e.g. exhalation and inhalation phases of breathing cycle) ending up in multiple reconstructed, phase-specific datasets. The latter approach enables to realize motion-compensation by warping all reconstructed datasets to one phase (e.g. by using non-rigid image registration). Such approaches, however, require a high degree of periodicity in the motion pattern.

The method of the present disclosure may comprise temporal binning of medical images acquired by the medical imaging device by assigning the medical images to specific breathing cycle phases and reconstructing multiple 3D images, each corresponding to a respective breathing cycle phase, particularly using intrinsic or extrinsic binning techniques, where the respective breathing cycle phases may be optionally specified within specific temporal window sizes, which result in effective pixel-based binning in the spatial domain of the reconstructed 3D images. In an example, the tracking device may track a marker, and a current breathing phase and a 3D image corresponding to the current breathing phase may be selected based on a correlation of a marker movement and a breathing phase, particularly for use in tool tracking, particularly, wherein the method comprises displaying an overlay of a tool model with the 3D image corresponding to the current breathing phase. According to the present disclosure, gated navigation may be provided by executing or providing a navigation function only for a subset of the breathing cycle phases, particularly only in one of the breathing cycle phases.

Binning and gating-related examples are provided below.

In an example for a method for tracking a tool, such as a needle (e.g. ablation device or biopsy needle or the like), in a spatial and temporal domain is provided in the following. In the example, 4D CBCT is acquired using intrinsic (e.g. using Amsterdam Shroud filter) and/or extrinsic (e.g. based on optical tracking markers using a tracking system mounted to the medical imaging device, e.g. a gantry-mounted tracking device) binning of projections, i.e. assignment of projections to specific breathing cycle phases (e.g., inhalation phase and exhalation phase) to reconstruct multiple CBCT datasets, each corresponding to a specific (and sensed) breathing cycle phase (e.g. inhalation, exhalation phase). Regardless of which projection binning method, intrinsic or extrinsic, is used-a correlation of marker movement (e.g. one or more markers attached to patient's chest-patient reference marker(s)) and the breathing phase is established/derived from the acquisition data, i.e., the X-ray projections and corresponding tracked marker positions. This allows deriving, at a later stage, in which breathing phase the patient currently is, for example just from observing the patient reference with the tracking device. Based on the above information (that is, the correlation describing the relationship between patient reference marker(s) movement and corresponding breathing cycle, and the 4D CBCT, i.e., the multiple 3D CBCTs), tool (e.g. needle) tracking (navigation) may be performed. The tool tracking may comprise that both, the tool (e.g. needle) and the patient reference, are tracked in the imaging system's coordinate system using the (e.g.) gantry-mounted tracking system, and based on this, the tool's model (e.g. CAD model) may be displayed on a screen on top of the acquired CBCT dataset (e.g. in a cross-sectional image view).

Contrary to known methods, not only spatial information based on sensed current patient and tool position is used for the display, but additionally the current breathing cycle phase of the patient may be obtained. Based thereon, the currently overlaid CBCT image on display may be replaced with the most appropriate CBCT image of the 4D CBCT set based on the breathing cycle phase. That is, for example, when sensing that the patient is currently in the inhalation phase, the CBCT (made of binned projections) of the inhalation phase may be used. As another example, when it is sensed that the patient is currently 30% after inhalation, but 70% before the exhalation phase, an accordingly interpolated CBCT (accordingly mixed by corresponding deformation vector interpolation between the phases) may be displayed as overlay image (one could compare this to a live “movie” being played, whose speed and current replay position is determined by the sensed breathing cycle surrogate). Additionally, for image noise reduction, e.g. in such interpolation scenarios, implicit pixel-based binning may be applied to the displayed CBCT. As an example, if the 30% inhalation phase is to be shown, all phases within a window having a window size, e.g. all phases from 20% to 40%, may be binned (i.e. averaged) in the displayed CBCT. The window size may be user-selectable. Selection of the window size is an effective means to control the trade-off between visible motion artefacts and visible pixel intensity noise.

Similarly to the above, spatial and temporal navigation might be realized in terms of a gated navigation. For example, gated navigation may entail that navigation is only available or executed in specific breathing cycles of the patient (e.g. only in the inhalation phase, which could also be denoted as deep inspiration breath hold technique).

The medical images I_i, according to the present disclosure, may be medical 2D projection images. The term “medical image” is therefore to be understood to particularly refer to medical 2D projection images.

The reference position, as used herein, refers to a reference position in C2, that is, a tracking position. A medical image at said reference position, in the present disclosure, may be referred to as the corresponding medical image or as the reference medical image.

In the present disclosure, a position C2 may also be referred to as a tracking position or a position in tracking coordinates.

Hereinbelow, the terms “tracking device” and “tracking system” are used interchangeably.

As an example, a medical imaging system may, for example, be an X-ray-based imaging system, such as a CT (computed tomography) imaging system. In particular, the medical imaging system may comprise a CBCT (cone beam CT).

As an example, a tracking device may be a camera, for example, infrared (IR) or in the visible spectrum, configured for tracking markers.

The tracking device may be detachably attached to the medical imaging system. The tracking device may be attached to the medical imaging system in a fixed relative position (including orientation) relative to a portion of the medical imaging system.

The tracking device may be configured to yield coordinates for the markers in the coordinate system C2 of the tracking device. The coordinate system C1 of the medical imaging system may, for example, have a fixed relation to C2, which may be determined in the course of the calibration.

As such, a transformation between coordinates in C1 and coordinates in C2 can be defined based on the calibration.

The medical imaging system may perform a scan. During the scan, the medical imaging system, the patient couch, or the subject might (inadvertently) change position in an unplanned manner, which has the potential of leading to motion artifacts.

As an example, a (residual) breathing motion of a patient, a sag of a patient couch, which particularly may happen gradually over time, coughing of the patient, flatulence may lead to motion artifacts.

Also saddle trajectory and helical scans may lead to motion artifacts unless motion correction is carried out.

Using the tracking device, such inadvertent motion can be accounted for.

Specifically, for each image, the tracking device may record tracking coordinates. From the perspective of the tracking device, the markers have changed position in the coordinate system C2, i.e., changed tracking coordinates.

The tracking coordinates are representative of the relative movement between the tracking device (and the medical imaging system) and the markers.

Based on the calibration and the tracking coordinates, specifically a detected motion, a transformation of the medical images may be carried out, the transformation, for example, being such that the medical images taken can be combined to reconstruct a 3D image with respect to a reference position from the transformed images accounting for any (intentional and/or unintentional) motion.

It will be understood that known marker-based tracking techniques may be used. Similarly, known transformation methods and reconstruction methods may be used.

The method may comprise determining the reference position and determining the reference position may comprise selecting one or more images among the plurality of medical images and determining the reference position based on the corresponding tracking positions of the selected image(s).

In particular, determining the reference position may comprise selecting an initial medical image I_1 as a reference image and using the corresponding tracking position as the reference position or selecting the last medical image I_n as a reference image and using the corresponding tracking position as the reference position.

That is, in a series of medical images, the first image may be the one used as a reference image and the corresponding tracking position as the reference position. This may be particularly advantageous where a real-time reconstruction is desired, in which case it might be detrimental to delay until further medical images have been captured. Alternatively, in a series of medical images, the last image may be the one used as a reference image and the corresponding tracking position as the reference position.

As another example, determining the reference position may comprise calculating a reference position based on a set of tracking positions corresponding to a set of medical images I_1, . . . I_x, particularly using statistical methods like determining a mean tracking position and using it as reference position.

As a further example, determining the reference position may comprise calculating a reference position based on one or more tracking positions at a specific time or within a specific time frame.

For example, determining the reference position may comprise calculating a reference position based on a set of tracking positions corresponding to a certain timeframe, e.g. prior to medical imaging, particularly using statistical methods like determining a mean (tracking) position and using it as a reference position.

That is, in a series of medical images, the entire series or a sub-set thereof may be selected and used to obtain a reference position, for example taking into account the position of each of the selected medical images, or alternatively, independently of any medical images, a series of tracking positions from a certain time frame may be selected and used to obtain a reference position. For example, where an oscillating motion like breathing occurs during the scan, it may be possible to use the position around which the oscillation takes place as a reference position. Calculation of the mean may be limited to a subset of the possible degrees of freedom, e.g., only with respect to a specific translation direction.

Of course image reconstruction may also be done (for example at a later time) with a different reference position. Alternatively or in addition, a 3D image reconstructed using the reference position may be (e.g. rigidly) transformed so as to obtain a 3D image from a different reference position, i.e. without requiring a reconstruction with the different reference position.

Reconstructing the 3D images may be performed in real-time and the initial imaging system coordinate is used as reference position.

As briefly mentioned above, in real time applications an inacceptable delay might be brought about if using coordinates obtained at a later time.

The method of the present disclosure may comprise storing, with the medical images, the respective imaging system coordinates and corresponding tracking coordinates of the one or more markers.

This may allow to reconstruct the 3D images at a later time and with respect to different reference positions.

The method of the present disclosure may comprise storing, with the reconstructed 3D images, the respective reference position. Thus, subsequent analysis of the 3D images may be improved.

The method of the present disclosure may further comprise reconstructing second 3D images from the transformed images using a second reference position that is different from the reference position.

This may be done concurrently or the second 3D images may be constructed at a later time, particularly, where the respective coordinates are stored with the medical images.

The method of the present disclosure may comprise reconstructing the 3D images with respect to a reference position determined from a marker attached to a patient in proximity of the imaged region of interest (ROI) such that the tracked markers are a valid surrogate signal for any motion occurring in the ROI during acquiring the medical images (i.e., during imaging).

The method of the present disclosure may comprise utilizing the tracking device for tracking a tool used in a medical procedure in the imaging coordinate system.

That is, a tool may also be tracked using the tracking device, e.g. via markers attached to the tool. A tool position may be obtained in the tracking device's coordinate system and optionally, based on the calibration, it may then also be reflected in the coordinate system of the medical imaging device. Thereby, for example, a visualization of the tool with correct spatial relations, e.g. for navigation purposes, may be enabled.

According to the present disclosure, the tool may be a pointer tool and the tracking of the tool may be used for visualizing the pointer tool with respect to medical images obtained prior to the medical procedure and/or during the medical procedure, particularly for use in pointer planning workflows.

A pointer tool may be tracked and the medical imaging device may be arranged so as to adjust, based on the calibration between C1 and C2, the current projection geometry with respect to a direction indicated by the pointer tool.

The method of the present disclosure may comprise using a shared reference marker for acquiring the tracking coordinates and for tracking by means of a tracking camera of a surgical navigation system, the surgical navigation system performing registration of image data obtained by the surgical navigation system during the medical procedure with medical image data obtained in advance of the medical procedure so as to allow for navigation during the medical procedure.

Surgical navigation systems are often provided separately from medical imaging systems, as they can be used for a wide variety of applications. It is advantageous to allow for making use of the capabilities of these navigation systems together with the capabilities of the tracking device. The method proposed herein allows for doing so in a reliable and efficient manner.

According to the present disclosure, the shared reference marker may be selected in such a manner as to avoid artifacts in reconstructed images and to provide accurate registration of the medical image data obtained in advance of the medical procedure and/or image data obtained during the procedure.

The method of the present disclosure may comprise the surgical navigation system providing a time of registration to the medical imaging system, e.g. as a signal, and the medical imaging system using a position associated with the time of registration as a reference position for motion compensation in reconstructing the 3D images or deriving a reference position from multiple tracking positions within a time frame of registration.

This allows for increased reliability, as changes may occur after the time of registration, which might significantly impact the accuracy.

According to the present disclosure, the medical imaging system may be a wheeled system that is autonomously movable and the method may comprise using the tracking device for providing positional awareness of the medical imaging system.

Particularly, the medical imaging system may be configured to allow acquiring medical images for saddle trajectories.

As an example, the medical imaging system may be configured such that several degrees of freedom are possible, including one or more of C-arm tilt, gantry-tilt, C-arm-yaw, gantry-yaw, longitudinal translational movements of the gantry or C-arm. A trajectory of the gantry or C-arm combining two or more of these motions of the gantry or C-arm may be referred to as saddle trajectories.

Even without such trajectories, calibration is challenging, e.g., due to gravity and sag that might lead to deviations from nominal trajectories. Combined motions like saddle trajectories add complexity and uncertainty (e.g. due to influences from the floor on which the system moves) in terms of the actual trajectory (e.g., as compared to a nominal trajectory). Thus, projection geometry inherently may not be known very precisely.

The method according to the present disclosure, particularly using the tracking device together with a static marker, e.g. attached to a floor or patient bed, and/or a marker attached to a patient, allows for addressing such challenges, e.g., allows for efficient motion compensation when scanning using trajectories like the saddle trajectories.

Specifically, this allows for accurately determining relative movements accurately in the same manner as the motion compensation that does not involve motions except for source and detector rotation.

Where static markers, e.g. attached to a floor or patient bed allows for highly focused images, particularly when the subject does not move, and where the markers are attached to a patient, motion compensation and very accurate determination of projection geometry can be achieved at the same time.

In general, tracking an autonomously movable system is important for precise image reconstruction. Since the method proposed herein is a high-accuracy method for determining a position relative to markers, by using suitably arranged markers the medical imaging system, via the tracking device, may obtain accurate positional awareness even in challenging arrangements and relative to a position that can simply be selected by respective marker placement.

According to the present disclosure, the medical imaging system may comprise a tiltable gantry or a tiltable C-arm, particularly a gantry configured such that its rotation plane is tiltable or a C-arm configured such that its rotation plane is tiltable.

An example for such an imaging system is the Loop-X. In general, medical imaging systems allow for rotation of the source and detector in a plane. In addition, some imaging systems allow for a translational movement, e.g., in a direction perpendicular to the plane. According to the present disclosure, the gantry or C-arm may be configured and mounted such that the plane itself may be tilted. For example, the gantry may rotate around an axis that is parallel to the longitudinal axis of a patient bed. The gantry, in addition, may describe a translational movement in a direction parallel to the longitudinal axis. The gantry may also tilt around a horizontal axis that is perpendicular to the longitudinal axis, e.g., towards and against the translational movement direction.

For example, a tilt rotation of the C-arm or gantry may be a rotation around hinges used for mounting the C-arm or gantry, e.g., mounted to a fixed or movable support structure like one or more feet, particularly a support structure movable on wheels.

According to the present disclosure, the medical imaging system may comprise a gantry or a C-arm, particularly a tiltable gantry or a tiltable C-arm as described above, and the medical imaging system may be configured such that the gantry or the C-arm are movable to describe a yaw rotation. For example, a yaw rotation may be achieved by a wheeled support structure to which the C-arm or gantry is mounted, wherein particularly the wheeled support structure may be driven via traction, e.g., using back wheels. As an example, all wheels might be set to a 45 ° so as to form a circle yaw rotation.

According to the present disclosure, the tracking device may be rigidly attached to a component of the medical imaging system that is fixed within the imaging coordinate system or to a component of the imaging system that is movable within the imaging coordinate system.

That is, the medical imaging system may comprise components that are movable with respect to each other and the tracking device may be rigidly attached to one of said components. As an example, the imaging coordinate system C1 may be selected such that the tracking device coordinate system C2 remains in a fixed relation. Alternatively, the relation between the coordinate system C1 and C2 may not have a constant fixed relation, in which case, in order to being able to reconstruct the 3D image, a respective relation between the coordinate systems C1 and C2 is tracked, e.g., based on the control of the movement.

According to the present disclosure, the medical imaging system may comprise a radiation source and a radiation detector that are movable relative to each other, wherein the tracking device is rigidly attached to a medical imaging system so as to have a fixed spatial relation with respect to one of the radiation source and the radiation detector. The relation between coordinate systems C1 and C2 is then a function of the current source or detector position (angle), which may be pre-calibrated.

This may allow for high flexibility in terms of imaging geometries, while also allowing for precise 3D image reconstruction using the tracking device.

According to the present disclosure, the medical imaging system may comprise a radiation source and a radiation detector that have a fixed relative spatial position, wherein the tracking device is rigidly attached to a medical imaging system so as to have a fixed spatial relation with respect to the radiation source and the radiation detector. The relation between coordinate systems C1 and C2 is then a function of either the current source or detector position (angle), which may be pre-calibrated.

While this (i.e. the fixed relative relationship of radiation source and detector) is a bit less flexible in terms of imaging geometries, it may reduce calibration complexity.

According to the present disclosure, the tracking device may be configured for pose tracking and/or may comprise at least one of: an (e.g. near) infrared tracking system, particularly comprising one or more cameras, a video camera tracking system comprising one or more cameras, an electromagnetic tracking system. Said tracking systems may, for example, be monoscopic or stereoscopic tracking systems.

According to the present disclosure, the one or more markers may comprise markers attached to a patient couch and/or markers attached to the floor and/or markers attached to one or more portions of the patient, in particular a portion of the patient inside a region of interest and/or a portion of a patient, outside the region of interest of the medical imaging device.

Which markers to use may depend on a desired reference for reconstruction and/or positional awareness. Moreover, it may depend, as seen above, on whether a cooperation with a marker-based surgical navigation system is envisaged.

According to the present disclosure, the one or more markers may comprise a marker attached to a chest of a patient and/or a spine of a patient and/or a cranium of a patient and/or a marker attached to a limb of a patient.

Since the patient may move during a procedure, having markers attached to parts of the patient relevant for the imaging, e.g. near or in a region of interest, may improve reconstruction accuracy. That is, the perceived position of the imaging system relative to spatial fixed markers may then differ from the perceived position of the imaging system relative to markers in the region of interest. Taking both into account may allow for proper perspective for reconstruction and accounting for patient movement as well. This may be particularly relevant in the context of cooperation with a surgical navigation system which might be configured to work with markers attached to the patient.

According to the present disclosure, the one or more markers may additionally comprise markers attached to a treatment device, e.g. a radiation treatment device, and/or markers attached to a tool for use in a medical procedure.

This may allow tracking positions of said treatment devices and tools correctly within the coordinate systems of the tracking device and the medical imaging system.

According to the present disclosure, the one or more markers may be attached in such positions as to allow for at least one of: accounting for a tilt of a gantry or a C-arm, accounting for a sag of a gantry or a C-arm, accounting for a device yaw, motion compensation during a longitudinal scan, accounting for slip of wheels of the medical imaging system on the floor, accounting for an uneven floor beneath the medical imaging system, particularly when the medical imaging system is a wheeled system.

As such, the tracking camera may, for example, be used for position control of the medical imaging device, like an autonomously movable device (e.g. where very accurate positional awareness of the device is of interest). The medical imaging system may, for example, based on the tracking camera, carry out a guided motion using markers that are static or even movable markers that allow for a follow-function.

As mentioned above, according to the present disclosure, the medical imaging system may be a wheeled system that is autonomously movable and the method according to the present disclosure may comprise using the tracking device for providing positional awareness of the medical imaging system. In such a case, for example, the method according to the present disclosure may comprise the medical imaging system repositioning itself automatically to a target position, e.g. a medical imaging position, after having been moved from said target position, wherein repositioning comprises: acquiring, during the repositioning, images of one or more markers by means of the tracking device and determining current marker positions from said images; using previous marker positions determined from images of markers acquired by the tracking device before moving or being moved from the target position, the images of the markers and/or marker positions of the markers and/or marker IDs of the markers optionally stored with corresponding medical images; and based on the current marker positions and the previous marker positions, determining a trajectory for repositioning the medical imaging system.

The method of the present disclosure may comprise the tracking device attached to the medical imaging system acquiring images of markers positioned at fixed positions in a room and uses marker positions determined from said images for determining the medical imaging system's position. Alternatively, the method may comprise a fixed-position tracking device, e.g. attached to a wall or a ceiling or a floor of a room or an equipment in the room having a known position in the room, being used to track the position of the medical imaging system in the room by acquiring images of one or more markers attached to the medical imaging system with the fixed-position tracking device and determining the position of the medical imaging system based on marker positions in the acquired images. Yet alternatively, the tracking device attached to the medical imaging system and a fixed-position tracking device, e.g. attached to a wall or a ceiling or a floor of a room or an equipment in the room having a known position in the room, may each acquire images of the same one or more markers positioned at fixed positions in the room or dynamic markers and marker positions determined from said images may be used for determining the medical imaging system's position (e.g. in the room) by combining the marker position(s) determined using the medical imaging system and the marker position(s) determined using the fixed-position tracking device to yield the position of the medical imaging system in relation to the fixed-position tracking device. Optionally, the medical imaging system's position may be determined relative to a room coordinate system and/or relative to a tool having a position calibrated relative to the room coordinate system.

For some cases, the medical imaging device, particularly the autonomously movable device, (e.g. the Loop-X) must be moved away from the imaging position (e.g. after imaging) and back to it several times, e.g. for re-imaging (e.g. when clearing operating space for a clinician or surgeon). In these cases, it is advantageous (specifically for re-imaging) if the same positions of the imaging device as before are approached. This can be achieved if the medical imaging device can reposition itself accurately in the room. Since the accuracy of the basic repositioning of the medical imaging device based on its internal and intrinsic wheel-based coordinate system is influenced by the condition of the floor, it can lead to unsatisfying repositioning accuracy of the medical imaging device in case the conditions are not good. This issue, among others, may be addressed as follows.

A (e.g. gantry-mounted) tracking system mounted to the medical imaging device may, particularly using the gantry-mounted tracking camera, continuously detect optical markers placed in its field of view (FOV) to obtain marker data, and store the marker data in the corresponding X-ray projections. The stored optical marker data of the previously acquired image (i.e., before moving away from the imaging position) can be compared with the same optical tracking markers visible for the tracking system when repositioning. A corrective trajectory for the repositioning of the medical imaging device can then be calculated from this data, for example, can be calculated subtracting the currently sensed optical tracking marker position from the stored optical tracking marker position of the previously acquired image. This allows a highly accurate repositioning of the medical imaging device to reacquire images and better comparability between the taken images at different points in time. For the sake of completeness, it is assumed that the optical tracking markers used for this purpose are not moved between the image acquisition and the repositioning movement, which is typically the case for navigated workflows. The above can also prevent additional patient dose and save time because in case the basic repositioning is too far off, the acquisition of another image is required.

The gantry mounted tracking system of the present disclosure, e.g. as described above in detail, allows determining tool positions and/or relative motion of the patient (motion compensation) in the coordinate system of the imaging device.

Since the medical imaging device (e.g. Loop-X) may be a mobile imaging device, this does not necessarily give information of the tool or patient movement in an outer room coordinate system (i.e. if the medical imaging device is moved). Reference markers may be provided statically in the room (i.e. at a known fixed position in the room). Using these markers as reference allows for resolving this issue.

Alternatively or in addition, the imaging device's position in the room may be determined solely by a tracking system (for example using IR camera arrays) mounted statically to the room's ceiling or walls or floor or an (non-)moveable equipment located in the room at known position (referred to as room tracking system), for example in combination with markers attached to the medical imaging device, e.g. to the medical imaging device's gantry, the markers being captured by the room tracking system.

Alternatively, the imaging device's position in the room may be determined by combining the information gained by the tracking system mounted to the medical imaging device (e.g. the gantry-mounted tracking system) with one or more statically room-mounted tracking systems. In this example, alternatively or in addition to a static marker, a dynamic marker can be placed in the room, which is captured by both, the tracking system mounted to the medical imaging device (e.g. the gantry-mounted tracking system) and the room tracking system(s) during imaging. These two pieces of information allow exact localization of the medical imaging device in the room, and thereby also exact relation to other components and devices with known position in the room, e.g. fixed particle therapy beam. An advantage of a dynamic marker is that this marker's position can be situationally adjusted, as needed, thereby allowing to overcome different challenges.

Using a room tracking system (alone or in combination with tracking system mounted to the medical imaging device) allows to address challenges such as reliably capturing the medical imaging device's position in the room throughout the entire movement range of the device (e.g. yaw, translations) generally being very large.

The invention provides an image reconstruction method for a medical system comprising a medical imaging system and a tracking device, particularly attached to the medical imaging system, e.g. rigidly or in a movable manner, the method comprising calibrating a coordinate system C2 of the tracking device for marker-based tracking to a coordinate system C1 of the medical imaging system; performing (S12) a scan by means of the medical imaging system, wherein performing the scan comprises acquiring a plurality of medical images I_i (i=1 . . . n); by means of the tracking device, for each of the plurality of medical images Li, acquire corresponding tracking coordinates in C2 using one or more markers; transforming the medical images based on the tracking coordinates and the calibration between C1 and C2 ; and reconstructing 3D images based on the transformed images with respect to a reference position, which may for example be associated with a reference medical image.

The features described above apply accordingly to this method.

As mentioned above, the present disclosure also provides a medical system, computer program product, and computer readable medium according to the independent claims.

In particular, the present disclosure also provides a medical system comprising a medical imaging system and a tracking device attached, particularly rigidly attached, to the medical imaging system, the system configured to carry out the method of any of the preceding claims.

In particular, the present disclosure also provides a medical system comprising a medical imaging system and a tracking device, particularly attached to the medical imaging system, e.g. rigidly or in a movable manner, the system configured to carry out the method of any of the preceding claims.

According to the present disclosure, the medical system may further comprise a medical imaging system configured to perform the scan and a tracking device configured to acquire the corresponding tracking coordinates.

For example, the medical imaging system may comprise an X-ray based system, e.g., a CT scanner. Particularly, the medical imaging system may be a cone beam CT, CBCT. Even more particularly, the medical imaging system may be an autonomously movable system. The medical imaging system may have a tiltable gantry or C-arm.

The tracking device may comprise a camera, e.g., a monoscopic or stereoscopic camera. It may comprise an infrared camera and/or a camera using the visible spectrum or other EM spectra.

Optionally, the medical system may further comprise tracking markers for use by the tracking device, the tracking markers attached to one or more of a patient bed, one or more body parts of the patient, the floor, one or more tools for use in a medical procedure, one or more components of a treatment machine, e.g., a radiation treatment machine or histotripsy device. Potential configuration of markers will be described below in the terminology section.

According to the present disclosure, the medical system may further comprise a navigation system for medical procedures, the navigation system using one or more navigation markers, wherein at least a subset of the navigation markers correspond to at least a subset of the tracking markers, particularly to a subset of the tracking markers attached to the patient.

Thus, as explained above, a reliable and precise synergy of the capabilities of the navigation system and the medical imaging system can be achieved.

According to the present disclosure, the tracking device may comprise one or more cameras enclosed by a housing, particularly completely enclosed by a housing, wherein the housing is configured to seal off non-marker-related reflections from an image sensor of the one or more cameras, particularly reflections from integrated illumination sources.

The present disclosure also provides a computer program product comprising instructions which, when the program is executed by a computer, cause the computer to control and/or carry out any of the steps of the method according to the present disclosure, particularly of the method claims.

The present disclosure also provides a computer-readable medium comprising instructions which, when executed by a computer, cause the computer to control and/or carry out any of the steps of the method according to the present disclosure, particularly of the method claims.

The present disclosure also relates to the use of the method and/or system of the present disclosure for medical imaging and/or performing a navigation, a positioning and/or alignment of a subject and various objects, e.g., pieces of equipment or tools.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable to medical imaging, navigation, positioning and/or alignment. For this reason alone, no surgical or therapeutic activity and in particular no surgical or therapeutic step is necessitated or implied by carrying out the invention.

The features and advantages outlined above in the context of the method similarly apply to the medical system, computer program product and computer readable medium of the present disclosure.

### Definitions

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n-and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Registering

The n-dimensional image of a body is registered when the spatial location of each point of an actual object within a space, for example a body part in an operating theatre, is assigned an image data point of an image (CT, MR, etc.) stored in a navigation system.

### Image Registration

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

### Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

### Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

### Marker Holder

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

### Pointer

A pointer is a rod which comprises one or more-advantageously, two-markers fastened to it and which can be used to measure off individual co-ordinates, for example spatial co-ordinates (i.e. three-dimensional co-ordinates), on a part of the body, wherein a user guides the pointer (for example, a part of the pointer which has a defined and advantageously fixed position with respect to the at least one marker attached to the pointer) to the position corresponding to the co-ordinates, such that the position of the pointer can be determined by using a surgical navigation system to detect the marker on the pointer. The relative location between the markers of the pointer and the part of the pointer used to measure off co-ordinates (for example, the tip of the pointer) is for example known. The surgical navigation system then enables the location (of the three-dimensional co-ordinates) to be assigned to a predetermined body structure, wherein the assignment can be made automatically or by user intervention.

### Reference Star

A “reference star” refers to a device with a number of markers, advantageously three markers, attached to it, wherein the markers are (for example detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star for example features a way of being attached to the object (for example, a clamp and/or a thread) and/or a holding element which ensures a distance between the markers and the object (for example in order to assist the visibility of the markers to a marker detection device) and/or marker holders which are mechanically connected to the holding element and which the markers can be attached to.

### Navigation System

The present disclosure may be applied in the context of a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

The invention also relates to a navigation system for computer-assisted surgery, comprising:
a computer for processing the absolute point data and the relative point data;
a detection device for detecting the position of the main and auxiliary points in order to generate the absolute point data and to supply the absolute point data to the computer;
a data interface for receiving the relative point data and for supplying the relative point data to the computer; and
a user interface for receiving data from the computer in order to provide information to the user, wherein the received data are generated by the computer on the basis of the results of the processing performed by the computer.

### Surgical Navigation System

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

### Landmarks

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

### Imaging Geometry

The information on the imaging geometry preferably comprises information which allows the analysis image (x-ray image) to be calculated, given a known relative position between the imaging geometry analysis apparatus and the analysis object (anatomical body part) to be analysed by x-ray radiation, if the analysis object which is to be analysed is known, wherein “known” means that the spatial geometry (size and shape) of the analysis object is known. This means for example that three-dimensional, “spatially resolved” information concerning the interaction between the analysis object (anatomical body part) and the analysis radiation (x-ray radiation) is known, wherein “interaction” means for example that the analysis radiation is blocked or partially or completely allowed to pass by the analysis object. The location and in particular orientation of the imaging geometry is for example defined by the position of the x-ray device, for example by the position of the x-ray source and the x-ray detector and/or for example by the position of the multiplicity (manifold) of x-ray beams which pass through the analysis object and are detected by the x-ray detector. The imaging geometry for example describes the position (i.e. the location and in particular the orientation) and the shape (for example, a conical shape exhibiting a specific angle of inclination) of said multiplicity (manifold). The position can for example be represented by the position of an x-ray beam which passes through the centre of said multiplicity or by the position of a geometric object (such as a truncated cone) which represents the multiplicity (manifold) of x-ray beams. Information concerning the above-mentioned interaction is preferably known in three dimensions, for example from a three-dimensional CT, and describes the interaction in a spatially resolved way for points and/or regions of the analysis object, for example for all of the points and/or regions of the analysis object. Knowledge of the imaging geometry for example allows the location of a source of the radiation (for example, an x-ray source) to be calculated relative to an image plane (for example, the plane of an x-ray detector). With respect to the connection between three-dimensional analysis objects and two-dimensional analysis images as defined by the imaging geometry, reference is made for example to the following publications:
1. “An Efficient and Accurate Camera Calibration Technique for 3D Machine Vision”, Roger Y. Tsai, Proceedings of the IEEE Conference on Computer Vision and Pattern Recognition. Miami Beach, Florida, 1986, pages 364-374
2. “A Versatile Camera Calibration Technique for High-Accuracy 3D Machine Vision Metrology Using Off-the-Shelf TV Cameras and Lenses”, Roger Y. Tsai, IEEE Journal of Robotics and Automation, Volume RA-3, No. 4, August 1987, pages 323-344.
3. “Fluoroscopic X-ray Image Processing and Registration for Computer-Aided Orthopedic Surgery”, Ziv Yaniv
4. EP 08 156 293.6
5. U.S. Ser. No. 61/054,187

### Shape Representatives

Shape representatives represent a characteristic aspect of the shape of an anatomical structure. Examples of shape representatives include straight lines, planes and geometric figures. Geometric figures can be one-dimensional such as for example axes or circular arcs, two-dimensional such as for example polygons and circles, or three-dimensional such as for example cuboids, cylinders and spheres. The relative position between the shape representatives can be described in reference systems, for example by co-ordinates or vectors, or can be described by geometric variables such as for example length, angle, area, volume and proportions. The characteristic aspects which are represented by the shape representatives are for example symmetry properties which are represented for example by a plane of symmetry. Another example of a characteristic aspect is the direction of extension of the anatomical structure, which is for example represented by a longitudinal axis. Another example of a characteristic aspect is the cross-sectional shape of an anatomical structure, which is for example represented by an ellipse. Another example of a characteristic aspect is the surface shape of a part of the anatomical structure, which is for example represented by a plane or a hemisphere. For example, the characteristic aspect constitutes an abstraction of the actual shape or an abstraction of a property of the actual shape (such as for example its symmetry properties or longitudinal extension). The shape representative for example represents this abstraction.

Referencing Determining the position is referred to as referencing if it implies informing a navigation system of said position in a reference system of the navigation system.

### Atlas/Atlas Segmentation

Preferably, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a patient's body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image-the atlas data therefore contains or is comparable to medical image data. Such a comparison can be carried out for example by applying an image fusion algorithm which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

### Analytical Devices

The movements of the treatment body parts are for example due to movements which are referred to in the following as “vital movements”. Reference is also made in this respect to EP 2 189 943 A1 and EP 2 189 940 A1, also published as US 2010/0125195 A1 and US 2010/0160836 A1, respectively, which discuss these vital movements in detail. In order to determine the position of the treatment body parts, analytical devices such as x-ray devices, CT devices or MRT devices are used to generate analytical images (such as x-ray images or MRT images) of the body. For example, analytical devices are constituted to perform medical imaging methods. Analytical devices for example use medical imaging methods and are for example devices for analysing a patient's body, for instance by using waves and/or radiation and/or energy beams, for example electromagnetic waves and/or radiation, ultrasound waves and/or particles beams. Analytical devices are for example devices which generate images (for example, two-dimensional or three-dimensional images) of the patient's body (and for example of internal structures and/or anatomical parts of the patient's body) by analysing the body. Analytical devices are for example used in medical diagnosis, for example in radiology. However, it can be difficult to identify the treatment body part within the analytical image. It can for example be easier to identify an indicator body part which correlates with changes in the position of the treatment body part and for example the movement of the treatment body part. Tracking an indicator body part thus allows a movement of the treatment body part to be tracked on the basis of a known correlation between the changes in the position (for example the movements) of the indicator body part and the changes in the position (for example the movements) of the treatment body part. As an alternative to or in addition to tracking indicator body parts, marker devices (which can be used as an indicator and thus referred to as “marker indicators”) can be tracked using marker detection devices. The position of the marker indicators has a known (predetermined) correlation with (for example, a fixed relative position relative to) the position of indicator structures (such as the thoracic wall, for example true ribs or false ribs, or the diaphragm or intestinal walls, etc.) which for example change their position due to vital movements.

### Treatment Beam

The present invention may also be employed in the field of controlling a treatment beam. The treatment beam treats body parts which are to be treated and which are referred to in the following as “treatment body parts”. These body parts are for example parts of a patient's body, i.e. anatomical body parts.

The present invention relates to the field of medicine and for example to the use of beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

Reference is also made in this respect to the following web pages: http://www.elekta.com/healthcare_us_elekta_vmat.php and http://www.varian.com/us/oncology/treatments/treatment_techniques/rapidarc.

### Arrangement of Treatment Beams

A treatment body part can be treated by one or more treatment beams issued from one or more directions at one or more times. The treatment by means of the at least one treatment beam thus follows a particular spatial and temporal pattern. The term “beam arrangement” is then used to cover the spatial and temporal features of the treatment by means of the at least one treatment beam. The beam arrangement is an arrangement of at least one treatment beam.

The “beam positions” describe the positions of the treatment beams of the beam arrangement. The arrangement of beam positions is referred to as the positional arrangement. A beam position is preferably defined by the beam direction and additional information which allows a specific location, for example in three-dimensional space, to be assigned to the treatment beam, for example information about its co-ordinates in a defined co-ordinate system. The specific location is a point, preferably a point on a straight line. This line is then referred to as a “beam line” and extends in the beam direction, for example along the central axis of the treatment beam. The defined co-ordinate system is preferably defined relative to the treatment device or relative to at least a part of the patient's body. The positional arrangement comprises and for example consists of at least one beam position, for example a discrete set of beam positions (for example, two or more different beam positions), or a continuous multiplicity (manifold) of beam positions.

For example, one or more treatment beams adopt(s) the treatment beam position(s) defined by the positional arrangement simultaneously or sequentially during treatment (for example sequentially if there is only one beam source to emit a treatment beam). If there are several beam sources, it is also possible for at least a subset of the beam positions to be adopted simultaneously by treatment beams during the treatment. For example, one or more subsets of the treatment beams can adopt the beam positions of the positional arrangement in accordance with a predefined sequence. A subset of treatment beams comprises one or more treatment beams. The complete set of treatment beams which comprises one or more treatment beams which adopt(s) all the beam positions defined by the positional arrangement is then the beam arrangement.

### Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography, as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

### Mapping

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### Elastic Fusion, Image Fusion/Morphing, Rigid

Image fusion can be elastic image fusion or rigid image fusion. In the case of rigid image fusion, the relative position between the pixels of a 2D image and/or voxels of a 3D image is fixed, while in the case of elastic image fusion, the relative positions are allowed to change.

In this application, the term “image morphing” is also used as an alternative to the term “elastic image fusion”, but with the same meaning.

Elastic fusion transformations (for example, elastic image fusion transformations) are for example designed to enable a seamless transition from one dataset (for example a first dataset such as for example a first image) to another dataset (for example a second dataset such as for example a second image). The transformation is for example designed such that one of the first and second datasets (images) is deformed, for example in such a way that corresponding structures (for example, corresponding image elements) are arranged at the same position as in the other of the first and second images. The deformed (transformed) image which is transformed from one of the first and second images is for example as similar as possible to the other of the first and second images. Preferably, (numerical) optimisation algorithms are applied in order to find the transformation which results in an optimum degree of similarity. The degree of similarity is preferably measured by way of a measure of similarity (also referred to in the following as a “similarity measure”). The parameters of the optimisation algorithm are for example vectors of a deformation field. These vectors are determined by the optimisation algorithm in such a way as to result in an optimum degree of similarity. Thus, the optimum degree of similarity represents a condition, for example a constraint, for the optimisation algorithm. The bases of the vectors lie for example at voxel positions of one of the first and second images which is to be transformed, and the tips of the vectors lie at the corresponding voxel positions in the transformed image. A plurality of these vectors is preferably provided, for instance more than twenty or a hundred or a thousand or ten thousand, etc. Preferably, there are (other) constraints on the transformation (deformation), for example in order to avoid pathological deformations (for instance, all the voxels being shifted to the same position by the transformation). These constraints include for example the constraint that the transformation is regular, which for example means that a Jacobian determinant calculated from a matrix of the deformation field (for example, the vector field) is larger than zero, and also the constraint that the transformed (deformed) image is not self-intersecting and for example that the transformed (deformed) image does not comprise faults and/or ruptures. The constraints include for example the constraint that if a regular grid is transformed simultaneously with the image and in a corresponding manner, the grid is not allowed to interfold at any of its locations. The optimising problem is for example solved iteratively, for example by means of an optimisation algorithm which is for example a first-order optimisation algorithm, such as a gradient descent algorithm. Other examples of optimisation algorithms include optimisation algorithms which do not use derivations, such as the downhill simplex algorithm, or algorithms which use higher-order derivatives such as Newton-like algorithms. The optimisation algorithm preferably performs a local optimisation. If there is a plurality of local optima, global algorithms such as simulated annealing or generic algorithms can be used. In the case of linear optimisation problems, the simplex method can for instance be used.

In the steps of the optimisation algorithms, the voxels are for example shifted by a magnitude in a direction such that the degree of similarity is increased. This magnitude is preferably less than a predefined limit, for instance less than one tenth or one hundredth or one thousandth of the diameter of the image, and for example about equal to or less than the distance between neighbouring voxels. Large deformations can be implemented, for example due to a high number of (iteration) steps.

The determined elastic fusion transformation can for example be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can for example be determined on the basis of a determined correlation between the first and second datasets.

### Fixed (Relative) Position

A fixed position, which is also referred to as fixed relative position, in this document means that two objects which are in a fixed position have a relative position which does not change unless this change is explicitly and intentionally initiated. A fixed position is in particular given if a force or torque above a predetermined threshold has to be applied in order to change the position. This threshold might be 10 N or 10 Nm. In particular, the position of a sensor device remains fixed relative to a target while the target is registered or two targets are moved relative to each other. A fixed position can for example be achieved by rigidly attaching one object to another. The spatial location, which is a part of the position, can in particular be described just by a distance (between two objects) or just by the direction of a vector (which links two objects). The alignment, which is another part of the position, can in particular be described by just the relative angle of orientation (between the two objects).

### Medical Workflow

A medical workflow comprises a plurality of workflow steps performed during a medical treatment and/or a medical diagnosis. The workflow steps are typically, but not necessarily performed in a predetermined order. Each workflow step for example means a particular task, which might be a single action or a set of actions. Examples of workflow steps are capturing a medical image, positioning a patient, attaching a marker, performing a resection, moving a joint, placing an implant and the like.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates a method according to the present disclosure;

FIG. 2 shows parts of a method according to the present disclosure; and

FIG. 3 is a schematic illustration of the system according to the present disclosure.

### DESCRIPTION OF EMBODIMENTS

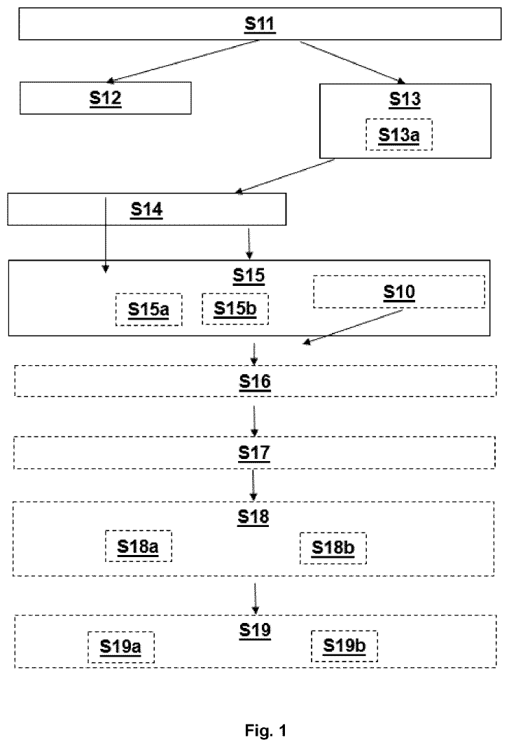

FIG. 1 illustrates the basic steps of an image reconstruction method for a medical system comprising a medical imaging system and a tracking device attached, particularly rigidly attached, to the medical imaging system according to the present disclosure. Alternatively, the tracking device may not be attached to the medical imaging system.

Step S11 encompasses calibrating a coordinate system C2 of the tracking device for marker-based tracking to a coordinate system C1 of the medical imaging system.

Subsequent step S12 encompasses performing a scan by means of the medical imaging system, wherein performing the scan comprises acquiring a plurality of medical images I_i (i=1 . . . n).

Step S13 encompasses, for each of the plurality of medical images I_i, by means of the tracking device, acquiring corresponding tracking coordinates in C2 using one or more markers.

The method may optionally comprise storing (S13a), with the medical images, the respective imaging system coordinates and corresponding tracking coordinates of the one or more markers.

The method may comprise determining, in optional step S10, the reference position. The reference position may, for example, be determined by selecting a medical image, for example the first medical image I_1, and using the corresponding tracking position as a reference position. Alternatively, the reference position may be determined by selecting some or all of the medical images and determining a corresponding mean tracking position and using the mean tracking position as a reference position.

Alternatively, the reference position may be based on a tracking position at a given time or a timeframe of tracking positions independently of medical image acquisition.

Step S14 encompasses transforming the medical images based on the tracking coordinates and the calibration between C1 and C2.

Step S15 encompasses reconstructing 3D images based on the transformed images with respect to a/the reference position. Reconstruction may be performed at a first time in step S15a, for example in real time and, for example, using the first image I_1 as a reference image. Alternatively or in addition, reconstruction may be carried out at a later time in step S15b, optionally with respect to a different reference position. The method may comprise storing, in optional step S16, with the reconstructed 3D images, the respective reference position.

The method may also comprise optional step S17 of tracking a tool, for example a pointer tool, by means of the tracking device, e.g. for visualization and/or selecting a reference position.

Optional step S18 may entail that a surgical navigation system carries out marker-based tracking concurrently with the tracking system attached to the medical imaging device carrying out the tracking.

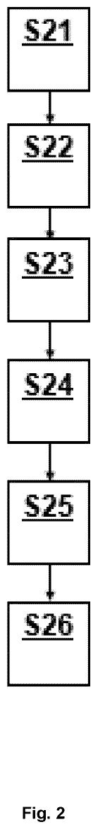

In this example, the method may comprise step S18a of the surgical navigation system using a shared reference marker that is also used by the tracking device attached to the medical imaging system and the step S18b of performing registration of image data obtained during the medical procedure and pre-procedural image data. In optional step S19, pointer planning may be carried out. That is, the medical imaging device may be positioned/aligned based on a pointer being tracked by means of the tracking device and then, optionally a planar X-ray image is acquired (step S19a) or a CBCT imaging is carried out (step S19b) from said direction/in said position. The step 19b may entail reverting to step S11. FIG. 2 illustrates in detail one example of transformation and 3D reconstruction. This may be employed in the method described in the context of FIG. 1.

In step S21, the coordinate system C1 of the imaging system is defined. For example, it may be defined with respect to the imaging geometry.

In step S22, the coordinate system C2 of the rigidly attached tracking device/system is defined. For example, the coordinate system may be defined with respect to the viewing direction of tracking camera(s) of the tracking device.

The order and timing of these steps can be selected freely.

In step S23, the coordinate systems C1 and C2 may be calibrated. This may entail determining the transformation that transforms a position in one of the coordinate systems to a position in the other of the coordinate systems. Specifically, as the tracking coordinates are obtained in C2, the transformation for representing a position or motion (described by the tracking coordinates) in C1 may be determined.

In Step S24, Medical Imaging Data Having Their Spatial Information in Coordinate system C1 are obtained.

At the same time, in step S25, tracking device tracks the position of markers in coordinate system C2, thereby determining relative motion. A synchronization mechanism between the tracking device and the imaging device ensures that the tracked position of markers can be unambiguously associated with the correct medical (projection) imaging data frames. If for one medical (projection) image data frame no association with a tracked position of markers is possible, the tracked position of markers of the previous frame shall be used.

In step S26, the medical (projection) imaging data along with the associated position of markers (transformed into C1 through calibration established in S21) are used for 3D image reconstruction.

For each medical (projection) image, its spatial information (S24) is modified by transforming its projection geometry prior to 3D reconstruction. The transformation is determined by subtracting the reference position of markers from the sensed tracked position of markers of the medical (projection) image.

Alternatively, for each medical (projection) image, its original spatial information (S24) is used for 3D reconstruction. However, in this case, the position (including orientation) of the 3D reconstruction is modified by the inverse of the abovementioned transformation prior to each reconstruction step.

Finally, S26 yields a motion-compensated 3D reconstruction, where the tracked marker positions of the underlying medical (projection) images were incorporated.

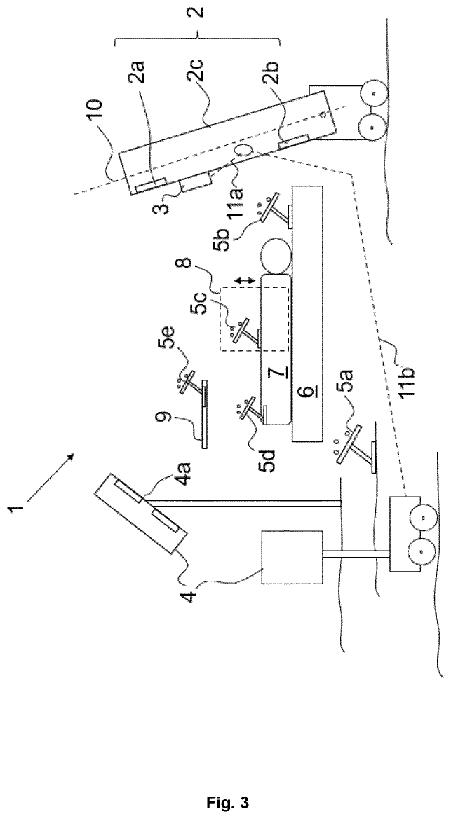

FIG. 3 discloses a medical system 1 according to the present disclosure. The medical system may be configured to carry out the methods of the present disclosure, particularly as outlined in the context of FIGS. 1 and 2.

The medical system comprises a medical imaging system 2, e.g. a CT imaging system. As an example, a Loop-X imaging system may be employed. An exemplary source 2a and exemplary detector 2b arranged on a gantry 2c are shown. The gantry may have a loop shape. The medical system further comprises a tracking device 3, in this example an infrared tracking camera. The tracking device is attached, particularly rigidly attached, to the medical imaging system 3, optionally in a detachable manner. Alternatively, the tracking device may not be attached to the medical imaging system.

The Figure also illustrates that the medical system may optionally comprise a surgical navigation system 4. Markers 5a to 5e are also illustrated, respectively arranged on the floor, attached to a patient bed 6, attached to a portion of the patient 7 inside the region of interest 8 or attached to a portion of the patient outside the region of interest, or attached to a medical tool 9. It is to be understood that any single one of and any combination of at least some of the markers 5a to 5e may be employed.

A tracking device of the surgical navigation system is denoted by reference sign 4a.

The medical imaging system is illustrated as a wheeled system and as a system having a tiltable gantry 2c, particularly the gantry having a tiltable rotation plane (which is in this illustration viewed from the side) indicated by dashed line 10.

The tracking device 3 and the medical imaging system 2 may communicate via data connection 11a. The surgical navigation system and the medical imaging system may communicate via a data connection 11b. The respective data connection may be wired or wireless.

The floor is shown as having some unevenness. This is merely for illustrating challenges in terms of position tracking of the medical imaging device. Other difficulties may arise from slip of the wheels on a potentially wet floor. These challenges can be overcome by the method and system of the present disclosure.

In the following, more examples and advantages will be described to illustrate the methods and systems of the present disclosure.

According to the present disclosure, an imaging system, such as a CT system, with a coordinate system (C1) and a tracking device, such as a tracking camera, rigidly attached to imaging system (C2), particularly a component thereof that is a fixed component relative to C1 or a mobile component relative to C1, are provided. For example, the tracking device may comprise a camera such as an optical stereoscopic camera, a monoscopic camera, an EM tracking device, or an infrared, camera. The tracking device may be attached, for example to a gantry or a C-arm to which source and/or detector of the imaging system are attached.

Particularly, the tracking device may be attached to the imaging system in such a manner that the relation between C1 and C2 is well-defined. Even more specifically, the tracking device may be attached in such a manner that if the gantry or C-arm tilts, the tracking camera tilts together with the gantry or C-arm, such that the relation between C1 and C2 remains constant.

As an example, C2 is calibrated to C1, optionally via another coordinate system like C3, for example an intrinsic calibration of detector or source relative to C1.

Thus, optical coordinate system C2 and radiological coordinate system C1 are cross-calibrated, particularly, for example, using a static cross-calibration.

Markers to be used by the tracking device may be positioned statically (e.g. on a patient couch or on the floor) or on a patient, particularly close to or removed from moving body parts. Markers may be positioned on the patient inside or outside the ROI of the imaging device.

Static mounting of markers may be used to address the challenge that for some trajectory types or device axes, accurate intrinsic calibration of an imaging device is difficult or not possible. For example, challenges arise in view of gantry tilt (e.g., due to residual varying gravity-induced sag), device yaw, or longitudinal/lateral scans and motion (due to slip of wheels and unpredictable floor surface). The imaging device may thus be tracked during a scan to improve image quality via more accurate “encoding” of the respective device axes.

Optionally, marker(s) is(are) positioned on a tool or treatment device, such as radiotherapy treatment device.

According to the present disclosure, (medical) images (I_i) may be acquired and stored/together with imaging system coordinates.

Tracking coordinates of (patient or static) marker(s) are acquired and stored with I_1

Tracking coordinates of (patient or static) marker(s) are acquired and store with I_2 and so on.

Image I_1 or another I_n (that may also be virtual and derived by statistical means such as the mean of a set of images I_x) is selected as image I_ref at a reference position.

All images I_i are transformed, for example using the transformation of trackingCoordinates(I_i)-trackingCoordinates(I_ref), giving I_i_transf.

As described above, alternatively, for each medical (projection) image, its original spatial information may be used for 3D reconstruction. However, in this case, the position (including orientation) of the 3D reconstruction is modified by the inverse of the abovementioned transformation prior to each reconstruction step.

A 3D image may be reconstructed based on transformed images I_i_transf

Reconstructed 3D image may be stored with respect to the selected/computed reference position.

As an example, a live reconstruction may be carried out based on I_1 as I_ref.

Optionally, a reference position can later be changed, e.g. average position, first, last, based on tracking of patient marker and similarity of current position to imaging position.

As described above, a combination with a surgical navigation system is conceivable. The surgical navigation system may have its own tracking camera and may perform independent registration of a pre-procedural image data set.

Without a tracking of the imaging system relative to the patient, intra-procedural image data may be blurry and/or misaligned to the pre-procedural data. Therefore, tracking of the imaging system relative to the patient with the same (a shared) reference marker (or with a marker rigidly attached to the reference) will provide intra-procedural image data that is correctly registered to the pre-procedural data and/or will not be blurry. “Blurry” in this context may generally denote a reconstructed image that shows image artefacts. This is consequently not ideal for navigation purposes and potentially introduces uncertainty and consequently inaccuracy into the navigation process.

A registration point in time may be provided as a signal from the surgical navigation system to the imaging system so that the imaging system can store and use this to determine the corresponding tracking position as a reference position for the motion correction, i.e., for the transforming of the medical images and performing 3D image reconstruction.

Optionally, the tracking device attached to the medical imaging system, e.g. on the gantry, may substitute or complement an external navigation system.

The tracking device may be used to track tools in the imaging coordinate system C1. Markers attached to the tools may be used to that end.

This may be employed for live visualization of pointer tools on intra-procedurally or pre-procedurally acquired images. Similarly, “pointer planning” workflows are possible. As an example, the medical imaging system may adjust the projection geometry based on a tracked pointer tool.

As another example, a treatment device such as a LINAC may have a marker attached to it and the patient motion correction marker may acts as reference for the substitute navigation system.

The tracking device may also be used for position control of the imaging system independent of imaging, e.g. where very accurate positional awareness of the imaging system is of interest.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1-27. (canceled)

28. An image reconstruction method for a medical system comprising a medical imaging system and a tracking device, the method comprising:
calibrating a coordinate system C2 of the tracking device for marker-based tracking to a coordinate system C1 of the medical imaging system;
performing, by the medical imaging system, a scan wherein the performing the scan comprises acquiring a plurality of medical images I_i (i=1 . . . n);
acquiring, by the tracking device, for each of the plurality of medical images I_i, corresponding tracking coordinates in the coordinate system C2 using one or more markers;
transforming the medical images based on the tracking coordinates and the calibration between the coordinate systems C1 and C2; and
reconstructing 3D images based on the transformed medical images with respect to a reference position.

29. The method according to claim 28, further comprising:
determining the reference position, wherein the determining the reference position comprises:
selecting one or more medical images among the plurality of medical images and determining the reference position based on corresponding tracking positions of the selected one or more medical images.

30. The method according to claim 29, wherein the determining the reference position comprises:
selecting an initial medical image I_1 as a reference medical image and using the corresponding tracking position as the reference position; and/or
selecting a last medical image I_n as the reference medical image and using the corresponding tracking position as the reference position; and/or
calculating the reference position based on a set of tracking positions corresponding to a set of medical images I_1, . . . I_x; and/or
calculating the reference position based on one or more tracking positions at a specific time or within a specific time frame,
optionally, wherein the reconstructing the 3D images is performed in real-time and an initial imaging system coordinate is used as the reference position.

31. (canceled)

32. The method according to claim 28, further comprising:
storing, with the medical images, respective imaging system coordinates and corresponding tracking coordinates of the one or more markers; and/or
storing, with the reconstructed 3D images, the respective reference position; and/or
reconstructing second 3D images from the transformed medical images using a second reference position that is different from the reference position; and/or
reconstructing the 3D images with respect to the reference position, wherein the reference position is determined from a marker attached to a patient and disposed in proximity of an imaged region of interest for providing a valid surrogate signal for any patient motion in the imaged region of interest occurring during the acquiring of the plurality of medical images.

33-34. (canceled)

35. The method according to claim 32, further comprising:
tracking, by the tracking device for tracking a tool, the tool used in a medical procedure in the coordinate system C1 of the medical imaging system,
optionally, wherein the tool is a pointer tool and the tracking the tool is for visualizing the pointer tool with respect to medical images obtained prior to the medical procedure and/or during the medical procedure.

36. (canceled)

37. The method according to claim 28, wherein the method comprises:
using a shared reference marker for acquiring the tracking coordinates and for tracking by via a tracking camera of a surgical navigation system; and
registering, by the surgical navigation system of, image data obtained by the surgical navigation system during a medical procedure with medical image data obtained in advance of the medical procedure so as to allow for navigation during the medical procedure; and
optionally, selecting the shared reference marker for avoiding artifacts in the reconstructed 3D images and for providing accurate registration of the medical image data obtained in advance of the medical procedure and/or the image data obtained during the medical procedure.

38. (canceled)

39. The method according to claim 28, further comprising:
providing, via a surgical navigation system, a signal to the medical imaging system, wherein the signal indicates a time of registration, ; and
using, by the medical imaging system a tracking position associated with the time of registration as the reference position for motion compensation in reconstructing the 3D images or deriving the reference position from multiple tracking positions within a time frame of registration; and/or
using the tracking device for providing positional awareness of the medical imaging system, the medical imaging system being a wheeled system that is autonomously movable; and/or
the medical imaging system automatically repositioning itself to a target position after having been moved from said target position.
wherein the repositioning comprises:
acquiring, by the tracking device during the repositioning, images of one or more markers and determining current marker positions from said images;
using previous marker positions determined from the images of the one or more markers acquired by the tracking device before moving or being moved from the target position, and optionally, storing the images of the one or more markers, and/or the marker positions of the markers, and/or marker IDs of the markers with corresponding medical images; and
determining a trajectory for repositioning the medical imaging system based on the current marker positions and the previous marker positions.

40-41. (canceled)

42. The method of claim 28, comprising:
inacquiring, by the tracking device attached to the medical imaging system, images of markers positioned at fixed positions in a room, and determining, via marker positions determined from said images, the medical imaging system's position; or
intracking, by a fixed-position tracking device, having a known position in the room, the position of the medical imaging system in the room by acquiring images of the one or more markers attached to the medical imaging system with the fixed-position tracking device, and determining, based on marker positions in the acquired images, the position of the medical imaging system
inacquiring, by the tracking device attached to the medical imaging system and by a fixed-position tracking device having a known position in the room, images of the same one or more markers positioned at fixed positions in the room or dynamic markers, and indetermining, via marker positions determined from said images the medical imaging system's position by combining the marker position determined using the medical imaging system and the marker position determined using the fixed-position tracking device to yield the position of the medical imaging system in relation to the fixed-position tracking device; and
optionally, determining the medical imaging system's position relative to a room coordinate system and/or relative to a tool having a position calibrated relative to the room coordinate system.

43. (canceled)

44. The method of claim 28, further comprising:
temporal binning of the plurality of medical images acquired by the medical imaging device by:
assigning the medical images to specific breathing cycle phases; and
reconstructing multiple 3D images, each corresponding to a respective breathing cycle phase, using intrinsic or extrinsic binning techniques,
optionally, wherein the respective breathing cycle phases are specified within specific temporal window sizes, which result in effective pixel-based temporal binning in a spatial domain of the reconstructed 3D images. (Currently amended) The method of claim comprising:
tracking, by the tracking device a marker and a current breathing phase;
selecting a 3D image corresponding to the current breathing phase based on a correlation of a marker movement and a breathing phase; and
displaying an overlay of a tool model with the selected 3D image corresponding to the current breathing phase; and
optionally, providing gated navigation by executing or providing a navigation function only for a subset of the breathing cycle phases.

46. (canceled)

47. The method according to claim 28, wherein the medical imaging system comprises:
a tiltable gantry or a tiltable C-arm, and/or a gantry or a C-arm, and the medical imaging system is configured such that the gantry or the C-arm are movable to describe a yaw rotation; and/or
a radiation source and a radiation detector, wherein:
the radiation source and the radiation detector are movable relative to each other, and the tracking device is attached to the medical imaging system so as to have an at least partially fixed spatial relation with respect to at least one of the radiation source or the radiation detector; or
the radiation source and the radiation detector have a fixed relative spatial position, and the tracking device is attached to the medical imaging system so as to have an at least partially fixed spatial relation with respect to the radiation source and the radiation detector.

48. The method according to claim 28, wherein:
the tracking device is attached to a component of the medical imaging system that is fixed within the coordinate system C1 of the medical imaging system or to a component of the medical imaging system that is movable within the coordinate system C1 of the medical imaging system; and/or
the tracking device is configured for pose tracking; and/or
the tracking device comprises at least one of: an infrared tracking system, a video camera tracking system comprising one or more cameras, and/or an electromagnetic tracking system.

49-50. (canceled)

51. The method according to claim 28, wherein the one or more markers comprise:
markers attached to a patient couch and/or a floor and/or one or more portions of the patient, wherein the markers are attached to one or more portions of the patient inside a region of interest of the medical imaging device, and/or wherein the markers are attached to one or more portions of the patient outside the region of interest of the medical imaging device; and/or
a marker attached to any one or more of: a chest of the patient, a spine of the patient, a cranium of the patient, and/or a limb of the patient; and/or
further comprise markers attached to a treatment device and/or a tool for use in a medical procedure; and/or
the one or more markers are attached in such positions to allow for accounting for one or more of:
a tilt of a gantry or a C-arm;
a sag of the gantry or the C-arm;
a device yaw;
motion compensation during a longitudinal scan;
slip of wheels of the medical imaging system on a floor; and/or
an uneven floor beneath the medical imaging system.

52-58. (canceled)

59. A medical system for image reconstruction, comprising:
a medical imaging system configured to perform a scan;
a tracking device, operatively coupled to the medical imaging system and configured to acquire corresponding tracking coordinates, wherein the tracking device and the medical imaging system are communicatively coupled via a data connection; and
optionally, one or more tracking markers for use by the tracking device, wherein the one or more tracking markers are attached to one or more of: a patient bed, one or more body parts of a patient, a floor, one or more tools for use in a medical procedure, and/or one or more components of a treatment device,
the medical system being configured to:
calibrate a coordinate system C2 of the tracking device for marker-based tracking to a coordinate system C1 of the medical imaging system;
perform, by the medical imaging system, the scan which comprises acquiring a plurality of medical images I_i (i=1 . . . n);
acquire, by the tracking device, for each of the plurality of medical images I_i, corresponding tracking coordinates in the coordinate system C2 using one or more markers;
transform the medical images based on the tracking coordinates and the calibration between the coordinate systems C1 and C2; and
reconstruct 3D images based on the transformed medical images with respect to a reference position.

60. (canceled)

61. The system according to claim 59, further comprising:
a surgical navigation system for medical procedures, wherein the surgical navigation system and the medical imaging system are communicatively coupled via a data connection, the surgical navigation system using one or more navigation markers, wherein at least a subset of the navigation markers correspond to at least a subset of the tracking markers, the surgical navigation system configured to register image data obtained by the surgical navigation system during a medical procedure with medical image data obtained in advance of the medical procedure so as to allow for navigation during the medical procedure.

62. The system according to claim 59, wherein the tracking device comprises one or more cameras enclosed by a housing, wherein the housing is configured to seal off non-marker-related reflections from an image sensor of the one or more cameras.

63. (canceled)

64. A non-transitory computer-readable storage medium storing a computer program comprising program instructions that, when executed on at least one processor of computer or loaded onto the at least one processor of the computer, cause the computer to perform an image reconstruction method for a medical system comprising a medical imaging system and a tracking device, by:
calibrating a coordinate system C2 of the tracking device for marker-based tracking to a coordinate system C1 of the medical imaging system;
performing, by the medical imaging system, a scan, wherein the performing the scan comprises acquiring a plurality of medical images I_i (i=1 . . . n);
acquiring, by the tracking device, for each of the plurality of medical images I i. corresponding tracking coordinates in the coordinate system C2 using one or more markers;
transforming the medical images based on the tracking coordinates and the calibration between the coordinate systems C1 and C2; and
reconstructing 3D images based on the transformed medical images with respect to a reference position.

65. The system according to claim 61, wherein the medical system is further configured to:
provide, by the surgical navigation system, a signal to the medical imaging system, wherein the signal indicates a time of registration; and
use, by the medical imaging system, a tracking position associated with the time of registration as the reference position for motion compensation in reconstructing the 3D images or deriving the reference position from multiple tracking positions within a time frame of registration; and/or
provide, via the tracking device, positional awareness of the medical imaging system, the medical imaging system being a wheeled system that is autonomously movable; and/or
the medical imaging system configured to automatically reposition itself to a target position, after having been moved from said target position,
the medical imaging system automatically repositioning itself by:
acquiring, by the tracking device during the repositioning, images of one or more markers and determining current marker positions from said images;
using previous marker positions determined from the images of the one or more markers acquired by the tracking device before moving or being moved from the target position, and optionally, storing the images of the one or more markers, and/or the marker positions of the markers, and/or marker IDs of the markers with corresponding medical images; and
determining, based on the current marker positions and the previous marker positions, a trajectory for repositioning the medical imaging system.

66. The system according to claim 59, wherein the medical imaging system comprises:
a tiltable gantry or a tiltable C-arm or a gantry or a C-arm, wherein the medical imaging system is configured such that the gantry or the C-arm are movable to describe a yaw rotation; and/or
a radiation source and a radiation detector, wherein:
the radiation source and the radiation detector are movable relative to each other, and the tracking device is attached to the medical imaging system so as to have an at least partially fixed spatial relation with respect to at least one of the radiation source or the radiation detector; or
the radiation source and the radiation detector have a fixed relative spatial position, and the tracking device is attached to the medical imaging system so as to have an at least partially fixed spatial relation with respect to the radiation source and the radiation detector.

67. The system according to claim 59, wherein the tracking device is attached to a component of the medical imaging system that is fixed within the coordinate system C1 of the medical imaging system or to a component of the medical imaging system that is movable within the coordinate system C1 of the medical imaging system; and/or
wherein the tracking device is configured for pose tracking; and/or
wherein the tracking device comprises at least one of: an infrared tracking system, a video camera tracking system comprising one or more cameras, and/or an electromagnetic tracking system.
